(19) United States
(12) Patent Application Publication
(10) Pub. No.: US 2026/0257079 A1
(43) Pub. Date: Sep. 3, 2026

(54) ULTRASONIC DRIVE DEVICE

(71) Applicant: LG ELECTRONICS INC., Seoul (KR)

(72) Inventors: Sangwon KIM, Seoul (KR); Yongju YANG, Seoul (KR); Gyuseog HONG, Seoul (KR); Suwon SEO, Seoul (KR); Munseong KANG, Seoul (KR); Seungyoon SHIN, Seoul (KR); Dongwon KIM, Seoul (KR)

(73) Assignee: LG ELECTRONICS INC., Seoul (KR)

(21) Appl. No.: 18/994,830

(22) PCT Filed: Jul. 26, 2023
(86) PCT No.: PCT/KR2023/010822

§ 371 Date: Jan. 15, 2025

(30) Foreign Application Priority Data
Aug. 4, 2022 (KR) 10-2022-0097411

(51) Int. Cl. A61N 7/02; A61N 7/00

(52) U.S. Cl. CPC A61N 7/02; A61N 2007/0004; A61N 2007/0073; A61N 2007/0078

(57) ABSTRACT

An ultrasonic drive device comprises: a vibrator generating ultrasonic waves; a flexible circuit board electrically connected to the vibrator; and a control unit connected to the flexible circuit board to supply current to the vibrator and controlling the driving of the vibrator, wherein the control unit generates ultrasonic pulses of a first waveform having a first pulse repetition frequency of 50-200 mW/cm² intensity, and varies the duty ratio of the ultrasonic pulses of the first waveform to control the driving of the vibrator The ultrasonic drive device generates low-intensity ultrasonic pulses in the range of 50-200 mW/cm² to reduce heat generated by driving the vibrator, thus preventing low-temperature burns from occurring on a user's body during ultrasonic treatment. In addition, the ultrasonic drive device varies the duty ratio of ultrasonic pulses in a treatment frequency range to generate ultrasonic pulses of various waveforms so that ultrasonic waves can be delivered more efficiently to parts of the user's body, thereby lowering the intensity of the ultrasonic waves and improving the treatment effect.

### TECHNICAL FIELD

The present disclosure relates to an ultrasonic drive device for generating ultrasonic pulses of various waveforms by varying the duty ratio of a low-intensity ultrasonic pulse.

### BACKGROUND ART

Ultrasonic waves are a form of mechanical energy that is transmitted through living tissue and are widely used in the field of medicine as a therapeutic, diagnostic, and surgical tool. Therapeutic ultrasonic waves may be divided into high-intensity therapeutic ultrasonic waves of 1000 W/cm² or more and low-intensity therapeutic ultrasonic waves of 10 mW/cm² to 50 W/cm².

The low-intensity therapeutic ultrasonic waves may be divided into focused ultrasonic waves and pulsed ultrasonic waves, wherein the focused ultrasonic waves are mainly used for skin lifting by heating subcutaneous tissue to cause necrosis, and the pulsed ultrasonic waves are used in sports medicine and musculoskeletal therapy in order to reduce joint stiffness and muscle spasms, to heal fractures, and to regenerate cartilage cells.

In order to transmit the therapeutic ultrasonic waves into the body, an oscillator that generates ultrasonic waves and a member that contacts the body part are required, but heat generated by driving of the oscillator may be applied directly to the body, which may cause low-temperature burns to the user's body.

In addition, heat generation of the oscillator may change the characteristics of ultrasonic waves generated by the oscillator, which may have a detrimental effect on chondrogenesis.

Furthermore, the ultrasonic waves generated by the oscillators have the characteristic of being reflected or dissipated when coming into contact with air, and therefore the ultrasonic waves generated by the oscillator are lost as the ultrasonic waves are transmitted to the user's body, reducing the therapeutic effect.

Due to this problem, ultrasonic therapy using the low-intensity pulsed ultrasonic waves is limited in increasing the therapeutic effect while reducing the intensity of ultrasonic waves.

### DISCLOSURE

### Technical Task

The present disclosure provides an ultrasonic drive device, and more particularly, it is an object of the present disclosure to provide an ultrasonic drive device capable of generating a low-intensity ultrasonic pulse of 50 to 200 mW/cm² to lower heat generation by driving of an oscillator, thereby preventing low-temperature burns from being caused to a user's body during ultrasonic therapy, and generating ultrasonic pulses of various waveforms by varying the duty ratio of an ultrasonic pulse within a therapeutic frequency range such that ultrasonic waves are more efficiently transmitted to user's body, thereby improving the therapeutic effect while reducing the intensity of the ultrasonic waves.

Objects of the present disclosure are not limited to the aforementioned object, and other unmentioned objects will be clearly understood by a person having ordinary skill in the art to which the present disclosure pertains based on the following description.

### Technical Solutions

The present disclosure provides an ultrasonic drive device including an oscillator configured to generate ultrasonic waves, a flexible circuit board electrically connected to the oscillator, and a controller electrically connected to the flexible circuit board, the controller being configured to supply current to the oscillator and to control driving of the oscillator, wherein the controller generates an ultrasonic pulse of a first waveform having a first pulse repetition frequency with an intensity of 50 to 200 mW/cm² and varies the duty ratio of the ultrasonic pulse of the first waveform to control the driving of the oscillator.

The first pulse repetition frequency may be 1 to 3 MHz, and the duty ratio may be 20 to 80%.

The controller may control the driving of the oscillator to generate an ultrasonic pulse of a second waveform having a second pulse repetition frequency of 10 to 100 Hz, and the ultrasonic pulse of the first waveform may be included in the ultrasonic pulse of the second waveform.

The controller may control the driving of the oscillator to generate an ultrasonic pulse of a third waveform having a third pulse repetition frequency of 0.2 to 1 Hz, and the ultrasonic pulse of the second waveform may be included in the ultrasonic pulse of the third waveform.

The oscillator may be provided in plural so as to be electrically connected to the flexible circuit board, and the controller may control the driving of the plurality of oscillators.

The plurality of oscillators may be disposed in a polygonal shape.

The plurality of oscillators may be disposed in a triangular shape on each side of the controller.

The plurality of oscillators may be disposed in line in a longitudinal direction of the flexible circuit board while being spaced apart from each other.

The plurality of oscillators may be spaced apart from each other by different distances.

### Advantageous Effects

The present disclosure provides an ultrasonic drive device, and the ultrasonic drive device is capable of generating a low-intensity ultrasonic pulse of 50 to 200 mW/cm² to lower heat generation by driving of an oscillator, thereby preventing low-temperature burns from being caused to a user's body during ultrasonic therapy and preventing side effects that may be caused by changes in the characteristics of ultrasonic waves generated by the oscillator due to heat generation of the oscillator.

In addition, the ultrasonic drive device is capable of generating ultrasonic pulses of various waveforms by varying the duty ratio of an ultrasonic pulse within a therapeutic frequency range such that ultrasonic waves within the therapeutic frequency range are more efficiently transmitted to user's body, thereby improving the therapeutic effect while reducing the intensity of the ultrasonic waves.

In addition, a plurality of oscillators may be disposed in different shapes depending on a body contact area to concentrate ultrasonic waves on a treatment area, thereby enhancing the therapeutic effect using ultrasonic waves.

Effects of the present disclosure are not limited to the above effects, and other unmentioned effects will be clearly understood by a person having ordinary skill in the art to which the present disclosure pertains based on the following description.

### DESCRIPTION OF DRAWINGS

FIG. 1 is a perspective view showing a physical therapy device having an ultrasonic drive device according to an embodiment of the present disclosure.

FIG. 2 is a side view of the physical therapy device having the ultrasonic drive device according to the embodiment of the present disclosure.

FIG. 3 is an exploded perspective view of the physical therapy device having the ultrasonic drive device according to the embodiment of the present disclosure.

FIG. 4 is a view illustrating double insert molding of a silicone cover in the physical therapy device having the ultrasonic drive device according to the embodiment of the present disclosure.

FIG. 5 is a view showing a high-gloss polished rear surface of the silicone cover of the physical therapy device having the ultrasonic drive device according to the embodiment of the present disclosure.

FIG. 6 is a view showing a bending slit formed in a hydrogel of the physical therapy device having the ultrasonic drive device according to the embodiment of the present disclosure.

FIG. 7 is a view showing an SUS cap covering an upper surface of an oscillator in the physical therapy device having the ultrasonic drive device according to the embodiment of the present disclosure.

FIG. 8 is a view showing the layout of a plurality of oscillators in the physical therapy device having the ultrasonic drive device according to the embodiment of the present disclosure.

FIG. 9 is a view showing another embodiment of the layout of the plurality of oscillators in the physical therapy device having the ultrasonic drive device of the present disclosure.

FIG. 10 shows measurement data of ultrasonic waves superimposed according to the distance between the plurality of oscillators in the physical therapy device having the ultrasonic drive device according to the embodiment of the present disclosure.

FIG. 11 is a view showing the form in which the plurality of oscillators is disposed in line in the physical therapy device having the ultrasonic drive device according to the embodiment of the present disclosure.

FIG. 12 is a block diagram illustrating the configuration of an ultrasonic drive device of the present disclosure.

FIG. 13 is a view illustrating the duty ratio of an ultrasonic pulse of a first waveform in an ultrasonic drive device according to an embodiment of the present disclosure.

FIG. 14 is a view showing an ultrasonic pulse of the ultrasonic drive device according to the embodiment of the present disclosure.

FIG. 15 shows data on a change in the surface temperature of an oscillator over the drive time of the ultrasonic drive device according to the embodiment of the present disclosure.

FIG. 16 shows data on a change in the surface temperature over time of one knee of a hind leg of a rabbit to which the physical therapy device having the ultrasonic drive device according to the embodiment of the present disclosure is attached.

FIGS. 17 to 21 show data illustrating the therapeutic effect of the ultrasonic drive device according to the embodiment of the present disclosure.

### BEST MODE FOR DISCLOSURE

Description will now be given in detail according to exemplary embodiments disclosed herein, with reference to the accompanying drawings. The same or equivalent components may be provided with the same reference numbers, and description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

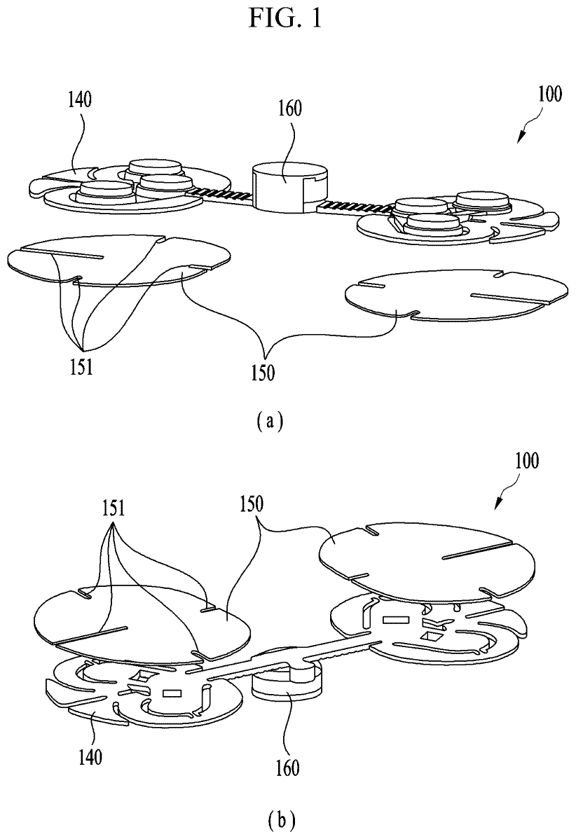

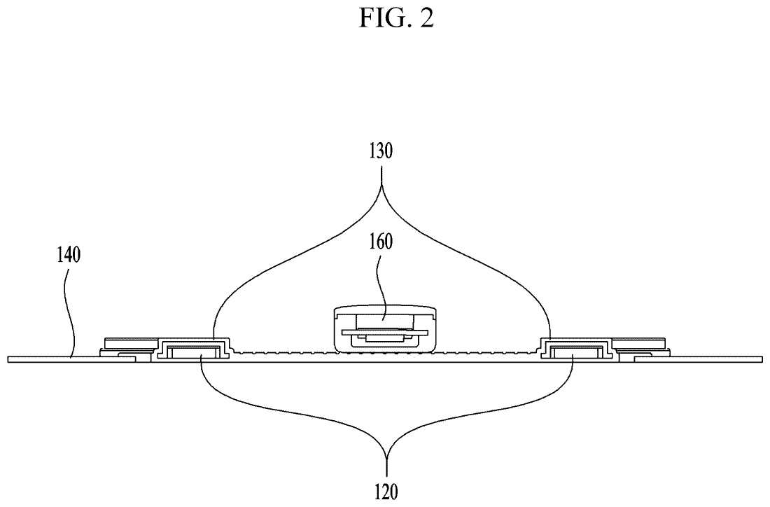

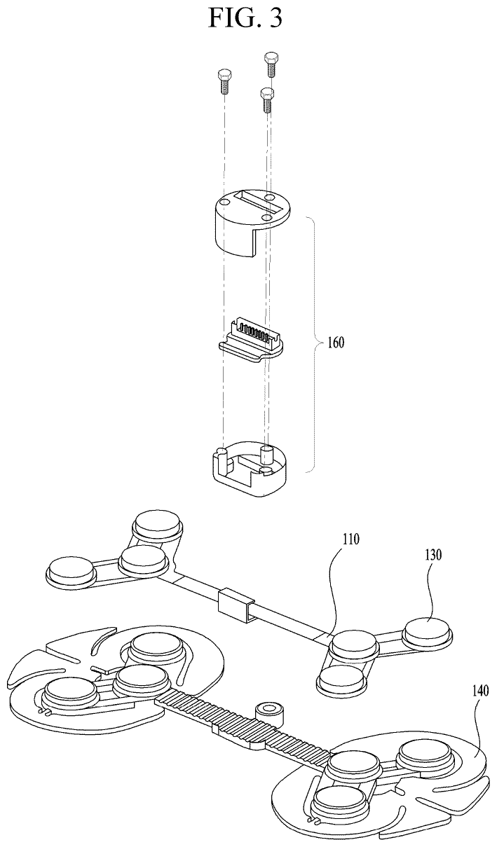

FIG. 1 is a perspective view showing a physical therapy device 100 having an ultrasonic drive device 300 according to an embodiment of the present disclosure. FIG. 2 is a side view of the physical therapy device 100 having the ultrasonic drive device 300 according to the embodiment of the present disclosure, and FIG. 3 is an exploded perspective view of the physical therapy device 100 having the ultrasonic drive device 300 according to the embodiment of the present disclosure.

The physical therapy device 100 having the ultrasonic drive device 300 of the present disclosure includes a flexible circuit board 110, a plurality of oscillators 120, an SUS cap 130, a silicone cover 140, a hydrogel 150, and a controller 160.

The plurality of oscillators 120 is electrically connected to the flexible circuit board 110, and the SUS cap 130 covers an upper surface of each of the plurality of oscillators 120. The silicone cover 140 covers the flexible circuit board 110, the plurality of oscillators 120, and the SUS cap 130.

The hydrogel 150 serves to allow ultrasonic waves generated by the plurality of oscillators 120 to penetrate a body contact area, and is detachably attached to a rear surface of the silicone cover 140. The controller 160 is connected to the flexible circuit board 110 to supply current to the plurality of oscillators 120. In particular, the rear surface of the silicone cover 140, to which the hydrogel 150 is detachably attached, is high-gloss polished.

In addition, the silicone cover 140 may have a thickness of 0.1 mm to 2 mm, taking into account durability and efficiency of ultrasound transmission.

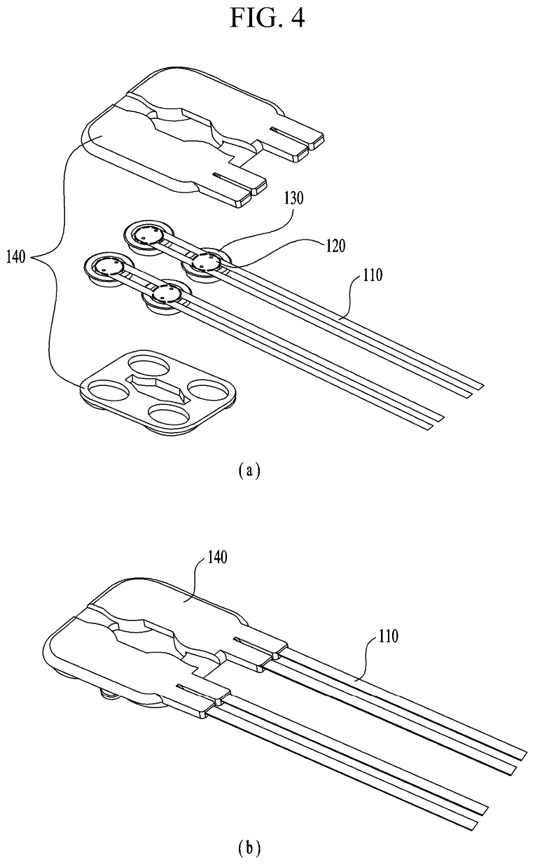

FIG. 4 is a view illustrating double insert molding of the silicone cover 140 in the physical therapy device 100 having the ultrasonic drive device 300 according to the embodiment of the present disclosure.

The silicone cover 140 is formed by hot pressing using a mold, and, the silicone cover 140 covers the flexible circuit board 110, the plurality of oscillators 120, and the SUS cap 130 by double insert molding. At this time, the adhesion of the silicone cover 140 to the flexible circuit board 110, the plurality of oscillators 120, and the SUS cap 130 may be performed using a primer.

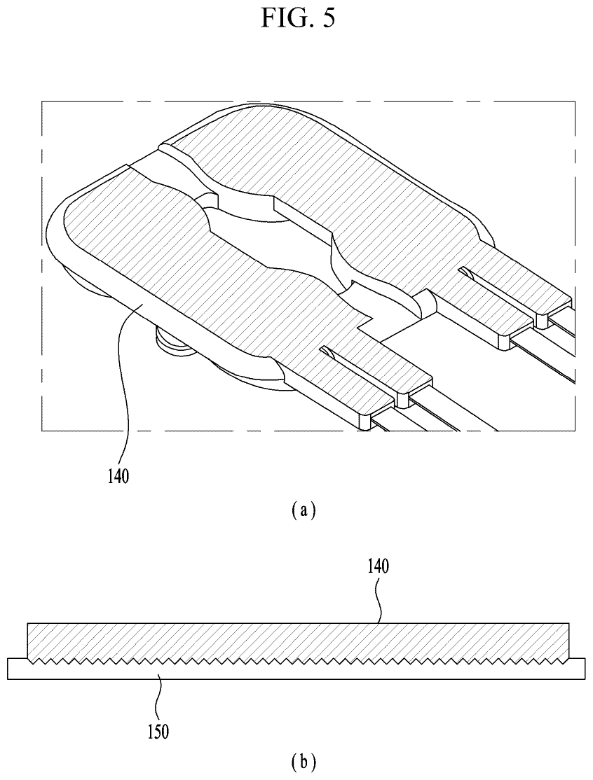

FIG. 5 is a view showing a high-gloss polished rear surface of the silicone cover 140 of the physical therapy device 100 having the ultrasonic drive device 300 according to the embodiment of the present disclosure.

FIG. 5(a) is a view showing the high-gloss polished rear surface of the silicone cover 140 when viewed from below, and FIG. 5(b) is a view showing that the hydrogel 150 is attached to the high-gloss polished rear surface of the silicone cover 140.

An ultrasonic input gel is generally used for ultrasonic waves to penetrate the body contact area. However, the ultrasonic input gel is not fixed to the body and flows off, resulting in the problem that the ultrasonic waves penetrate the body contact area only through the area where the ultrasonic input gel remains, whereby the therapeutic effect of the ultrasonic waves is reduced. Furthermore, when the ultrasonic input gel is used, it is necessary to clean the ultrasonic therapy device after use, which is inconvenient.

The physical therapy device 100 having the ultrasonic drive device 300 according to the present disclosure has a structure in which the hydrogel 150 is detachably attached to the rear surface of the silicone cover 140 without using an ultrasonic input gel. Since ultrasonic waves are reflected or dissipated when coming into contact with air, it is necessary to increase the adhesion between the rear surface of the silicone cover 140 and the hydrogel 150 such that there is no air layer between the rear surface of the silicone cover 140 and the hydrogel 150 in order to increase the therapeutic effect using ultrasonic waves.

In order to increase the adhesion to the hydrogel 150, it is necessary to ensure that the rear surface of the silicone cover 140 has a certain roughness rather than being flat. In the physical therapy device 100 having the ultrasonic drive device 300 of the present disclosure, therefore, the rear surface of the silicone cover 140 may be coated with a coating agent for surface treatment by high-gloss polishing in order to increase the adhesion to the hydrogel 150.

Referring to FIG. 5(b), the high-gloss polished rear surface of the silicone cover 140 has a certain roughness due to the formation of fine protrusions. This may increase the adhesion between the rear surface of the silicone cover 140 and the hydrogel 150, allowing ultrasonic waves to be transmitted to the body contact area without encountering air.

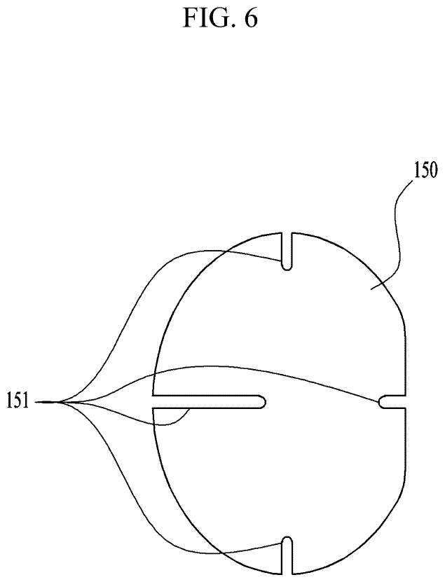

FIG. 6 is a view showing a bending slit formed in the hydrogel 150 of the physical therapy device 100 having the ultrasonic drive device 300 according to the embodiment of the present disclosure.

Conventionally, an ultrasonic input gel is used for ultrasonic waves to penetrate the body contact area, but the ultrasonic input gel is not fixed to the body and flows off, resulting in the problem that ultrasonic waves penetrate only the area where the ultrasonic input gel remains, whereby the therapeutic effect is reduced.

In the physical therapy device 100 having the ultrasonic drive device 300 according to the present disclosure, it is possible for ultrasonic waves to pass through the hydrogel 150 that is attached to the user's body without using the ultrasonic input gel. In addition, the hydrogel 150 may have at least one bending slit 151 extending from the periphery thereof, which may improve the flexibility of the hydrogel 150.

Even when attached to a curved body contact area such as the knee, therefore, the adhesion of the hydrogel may be increased to ensure uniform transmission of ultrasonic waves, and the restriction of movement may be minimized even in the state in which the physical therapy device 100 having the ultrasonic drive device 300 according to the present disclosure is attached to the user. Furthermore, since no ultrasonic input gel is used, there is no need for separate cleaning after treatment.

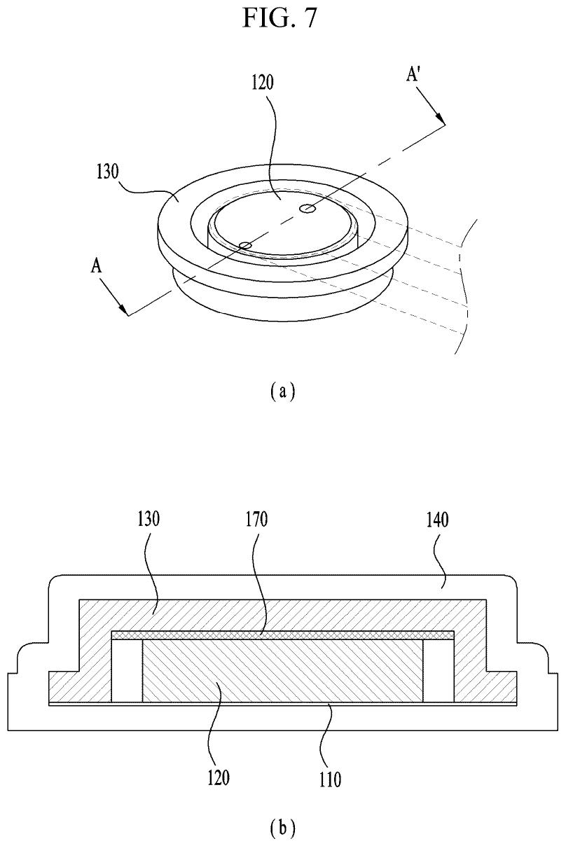

FIG. 7 is a view showing the SUS cap 130 covering the upper surface of the oscillator 120 in the physical therapy device 100 having the ultrasonic drive device 300 according to the embodiment of the present disclosure.

FIG. 7(a) is a bottom view of the SUS cap 130 covering the upper surface of the oscillator 120, and FIG. 7(b) is an A-A′ sectional view of FIG. 7(a).

The SUS cap 130 has a bonding layer 170 that abuts the upper surface of the oscillator 120, and is spaced apart from the side of the oscillator 120. The silicone cover 140 covers the flexible circuit board 110, the oscillator 120, and the SUS cap 130.

Referring to FIG. 7(b), the SUS cap 130 is spaced apart from the side of the oscillator 120, and has a bonding layer 170 that abuts the upper surface of the oscillator 120.

In order to enhance the therapeutic effect using ultrasonic waves, ultrasonic waves generated by the oscillator 120 must be focused onto the body contact area. To this end, the SUS cap 130 is provided with a bonding layer 170 that abuts the upper surface of the oscillator 120 such that some of the ultrasonic waves generated by the oscillator 120 that are directed to the opposite side of the body contact area can be reflected to the body contact area. The bonding layer 170 may be made of thin silicone.

Furthermore, the SUS cap 130 may also serve as a heat dissipation member in order to prevent heat generated by the plurality of oscillators 120 from being released to the outside.

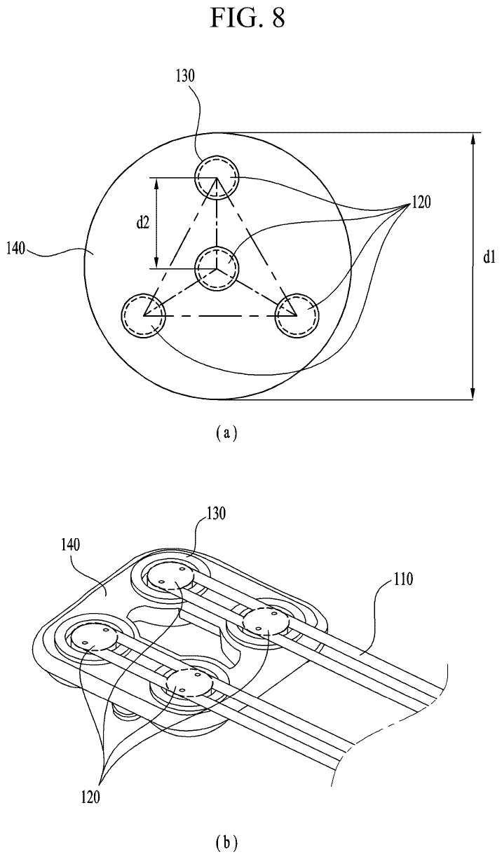

FIG. 8 is a view showing the layout of the plurality of oscillators 120 in the physical therapy device 100 having the ultrasonic drive device 300 according to the embodiment of the present disclosure.

FIG. 8(a) is a view showing the form in which the plurality of oscillators 120 is disposed in an equilateral triangular shape, and FIG. 8(b) is a view showing the form in which the plurality of oscillators is disposed in a quadrangular shape. This is the layout of the plurality of oscillators 120 for relieving muscle pain, such as back or lower back pain.

When the physical therapy device 100 having the ultrasonic drive device 300 is used to relieve muscle pain, it is necessary to ensure that ultrasonic waves generated by the plurality of oscillators 120 are evenly distributed over a large treatment area.

Referring to FIG. 8(a), the diameter d1 of the circular cylinder cover 140 may be 85 mm in consideration of ease of use. One oscillator 120 may be disposed in the center of the circular cylinder cover 140, and three oscillators 120 may be disposed in an equilateral triangle shape while having a distance d2 of 30 mm therefrom.

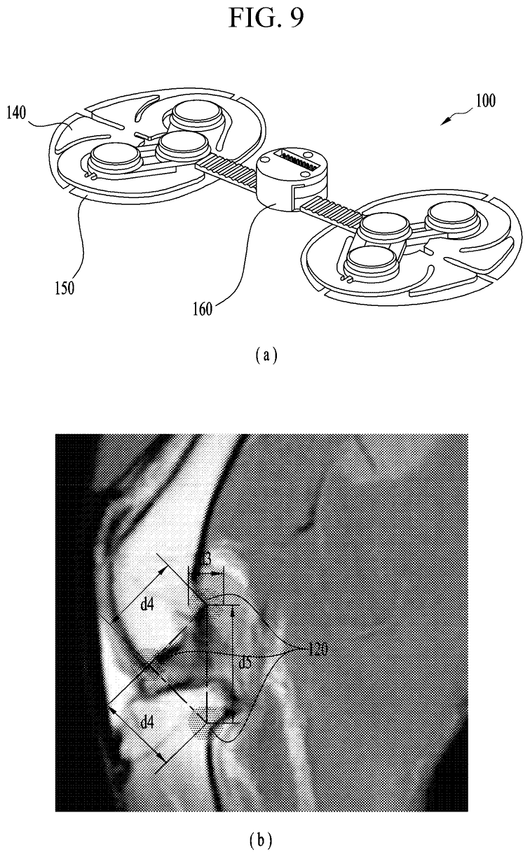

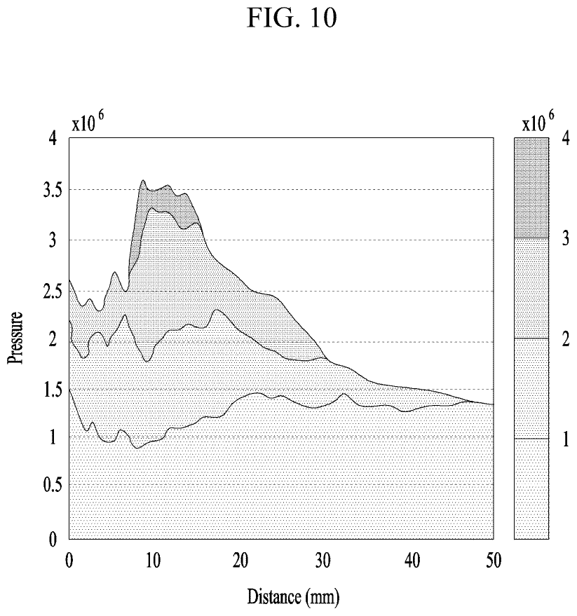

FIG. 9 is a view showing another embodiment of the layout of the plurality of oscillators 120 in the physical therapy device 100 having the ultrasonic drive device 300 of the present disclosure, and FIG. 10 shows measurement data of ultrasonic waves superimposed according to the distance between the plurality of oscillators 120 in the physical therapy device 100 having the ultrasonic drive device 300 according to the embodiment of the present disclosure.

FIG. 9(a) is a view showing the form in which the plurality of oscillators 120 is disposed in a triangular shape on each side of the controller 160. In this case, the physical therapy device 100 having the ultrasonic drive device 300 may be attached to the knee and used to treat arthritis. The controller 160 is located at the front of the knee, and the plurality of oscillators 120 disposed in a triangular shape on each side of the controller 160 is located on each side of the knee. FIG. 9(b) is a view showing the plurality of oscillators 120 disposed in a triangular shape on one side of the knee.

In order to increase the therapeutic effect by ultrasonic waves, ultrasonic waves generated by the plurality of oscillators 120 must be intensively transmitted to the cartilage in the knee. If the plurality of oscillators 120 is disposed at large intervals or if the plurality of oscillators is disposed at small intervals, the ultrasonic waves may not be intensively transmitted to the cartilage.

Referring to FIG. 10 illustrating this, it can be seen that, if the diameter of each of the plurality of oscillators 120 is 9 mm to 20 mm, ultrasonic waves are superimposed the most when the distance between the plurality of oscillators 120 is 9 mm to 20 mm.

In order to treat the arthritis of the knee, therefore, the plurality of oscillators 120 may be disposed in a triangular shape such that the diameter d3 of each of the plurality of oscillators 120 is 9 mm to 20 mm and the distances d4 and d5 between the plurality of oscillators 120 are 9 mm to 20 mm, as shown in FIG. 9. In this case, ultrasonic waves generated by the plurality of oscillators 120 may be intensively transmitted to the cartilage in the knee, thereby improving the knee arthritis treatment effect by ultrasonic waves.

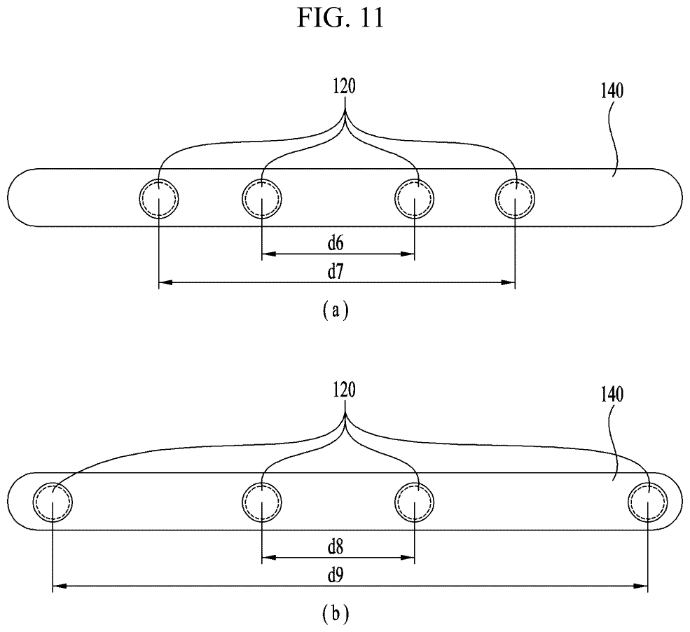

FIG. 11 is a view showing the form in which the plurality of oscillators 120 is disposed in line in the physical therapy device 100 having the ultrasonic drive device 300 according to the embodiment of the present disclosure.

FIG. 11(a) is a view showing the form in which the plurality of oscillators 120 is disposed in line while being spaced apart from each other in order to treat the arthritis of the wrist, and FIG. 11(b) is a view showing the form in which the plurality of oscillators 120 is disposed in line while being spaced apart from each other in order to treat the arthritis of the ankle.

The thickness of the ankle is greater than the thickness of the wrist, and the position of the cartilage is different accordingly. In consideration of this, it is necessary to dispose the plurality of oscillators 120 for treating the arthritis of the wrist and the ankle at different intervals.

For example, in the case of FIG. 11(a), the distance d6 between two oscillators 120 located on the inside may be 50 mm and the distance d7 between two oscillators 120 located on the outside may be 120 mm for treatment of the arthritis of the wrist.

In the case of FIG. 11(b), the distance d8 between two oscillators 120 located on the inside may be 50 mm and the distance d9 between two oscillators 120 located on the outside may be 210 mm for treatment of the arthritis of the ankle.

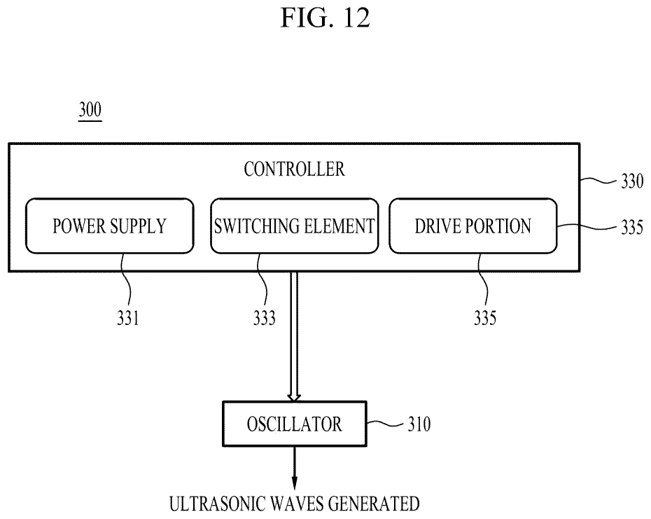

FIG. 12 is a block diagram illustrating the configuration of an ultrasonic drive device 300 of the present disclosure.

The ultrasonic drive device 300 of the present disclosure includes an oscillator 310, a printed circuit board 320, and a controller 330. The oscillator 310, which generates ultrasonic waves, is electrically connected to the printed circuit board 320, and the controller 330 is electrically connected to the printed circuit board 320 to supply current to the oscillator 310 and to control the driving of the oscillator 310. Here, the controller 330 includes a power supply 331, a switching element 333, and a drive portion 335.

The power supply 331 serves to turn on and off the power of the ultrasonic drive device 300. That is, when a user wishes to use the physical therapy device 100 having the ultrasonic drive device 300 of the present disclosure, the user may turn on the power to operate the ultrasonic drive device 300 in the state in which the physical therapy device 100 having the ultrasonic drive device 300 of the present disclosure is in contact with the body, and may arbitrarily turn off the power when the user completes use thereof.

In addition, the power supply 331 may serve as a timer that automatically turns off the power of the ultrasonic drive device 300 of the present disclosure when a preset treatment time is up. As a result, it is possible to increase stability of the use of the physical therapy device 100 having the ultrasonic drive device 300 and to prevent risks such as low-temperature burns of the user's body part.

The switching element 333 serves to vary the duty ratio of an ultrasonic pulse 340 of a first waveform having a first pulse repetition frequency, which is within a therapeutic frequency range. On and off of the switching element 333 may be controlled by the drive portion 335 such that the duty ratio of the ultrasonic pulse 340 of the first waveform can be varied. As such, the duty ratio of the ultrasonic pulse 340 of the first waveform may be varied by the switching element 333 and the drive portion 335 and the driving of the oscillator 310 may be controlled by the controller 330 such that ultrasonic pulses of various waveforms can be generated within the therapeutic frequency range and transmitted to the user's body.

In addition, by varying the duty ratio, the output voltage of the ultrasonic drive device 300 may be controlled within an ultrasonic output range, whereby power consumption may be reduced.

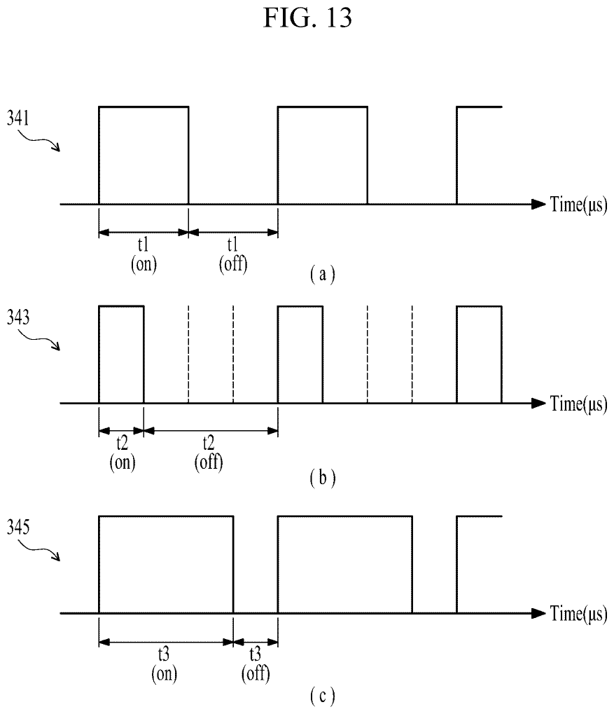

FIG. 13 is a view illustrating the duty ratio of an ultrasonic pulse 340 of a first waveform in an ultrasonic drive device 300 according to an embodiment of the present disclosure.

The duty ratio or the duty cycle is the percentage of time during which a pulse signal is on in one cycle of the pulse signal. Here, the cycle refers to the time necessary for a pulse signal to go through one complete on and off cycle.

FIG. 13(a) is a view illustrating an ultrasonic pulse 341 of a first waveform having a duty ratio of 50% in the ultrasonic drive device 300 according to the embodiment of the present disclosure. In this case, t1 (on):t1 (off) is a ratio of 1:1, and therefore the duty ratio, which is the percentage of time during which a pulse signal is on in one cycle of the pulse signal, is 50%.

FIG. 13(b) shows an ultrasonic pulse 343 of a first waveform having a duty ratio of 25% with a ratio of t2 (on):t2 (off) of 1:3. In addition, FIG. 13(c) shows an ultrasonic pulse 345 of a first waveform having a duty ratio of 75% with a ratio of t3 (on):t3 (off) of 3:1.

While FIG. 13 illustrates an example of a first waveform having the same period of the ultrasonic pulse 340 and different duty ratios in the ultrasonic drive device 300 of the present disclosure, the present disclosure not limited thereto. That is, it is possible to vary the period of the ultrasonic pulse 340 of the first waveform at the same duty ratio by controlling the time during which the pulse signal is on and the time during which the pulse signal is off.

Consequently, the ultrasonic drive device 300 of the present disclosure is capable of generating ultrasonic pulses of various waveforms by varying the duty ratio of the ultrasonic pulse 340 of the first waveform having a first pulse repetition frequency of 1 to 3 MHz, which is the therapeutic frequency range, to 20 to 80%. This may be realized by controlling on and off of the switching element 333 by the drive portion 335.

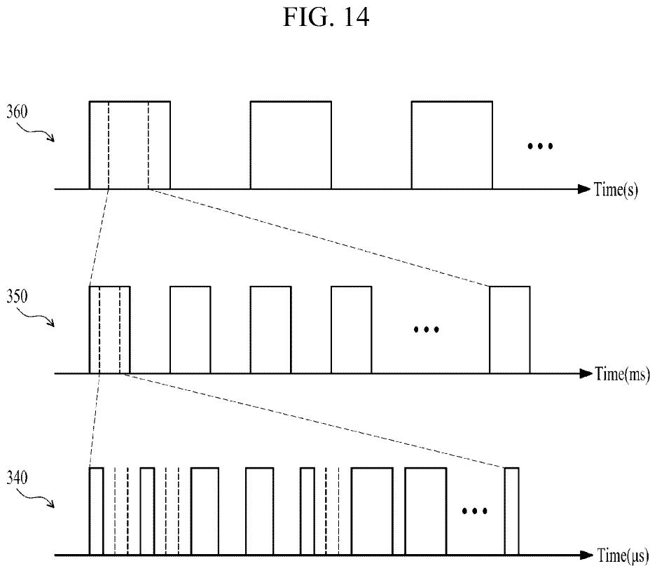

FIG. 14 is a view showing an ultrasonic pulse of the ultrasonic drive device 300 according to the embodiment of the present disclosure.

The ultrasonic drive device 300 of the present disclosure may generate an ultrasonic pulse 340 of a first waveform having a first pulse repetition frequency, which is within the therapeutic frequency range, and may vary the duty ratio of the ultrasonic pulse 340 of the first waveform. Here, the first pulse repetition frequency, which is within the therapeutic frequency range, may be 1 to 3 MHz, and the duty ratio may be varied to 20 to 80%.

An ultrasonic pulse 350 of a second waveform has a second pulse repetition frequency of 10 to 100 Hz. An ultrasonic pulse 360 of a third waveform has a third pulse repetition frequency of 0.2 to 1 Hz. Here, the ultrasonic pulse 350 of the second waveform having the second pulse repetition frequency and the ultrasonic pulse 360 of the third waveform having the third pulse repetition frequency may serve as an intermediate for lowering heat generation of the oscillator 310 in the ultrasonic drive device 300 of the present disclosure.

If the ultrasonic pulse 340 of the first waveform having a first repetition frequency of 1 to 3 MHz, which is within the therapeutic frequency range, is continuously generated during the treatment time, the above-described problem of heat generation of the oscillator may occur. In addition, heat generation of the oscillator may change the characteristics of ultrasonic waves generated by the oscillator, which may have a detrimental effect on chondrogenesis.

Therefore, in order to solve the problem of heat generation of the oscillator as described above, the ultrasonic pulse 340 of the first waveform may be included in the ultrasonic pulse 350 of the second waveform having a second pulse repetition frequency of 10 to 100 Hz such that the pulse repetition frequency is changed. In addition, the ultrasonic pulse 350 of the second waveform may be included in the ultrasonic pulse 360 of the third waveform having a third pulse repetition frequency of 0.2 to 1 Hz.

That is, the ultrasonic drive device 300 of the present disclosure may generate ultrasonic waves including all of the ultrasonic pulses 340, 350, and 360 of the first, second, and third waveforms with different pulse repetition frequencies and may transmit the same to the user's body. As a result, it is possible to increase the therapeutic effect using ultrasonic waves by reducing the intensity of ultrasonic waves to 50 to 200 mW/cm² but varying the duty ratio of the ultrasonic pulse 310 of the first waveform having the first pulse repetition frequency, which is within the therapeutic frequency range, to more efficiently transmit ultrasonic pulses of various waveforms to the user's body, and at the same time to reduce the heat generation of the oscillator 310 through the ultrasonic pulses 350 and 360 of the second and third waveforms.

Furthermore, the ultrasonic drive device 300 of the present disclosure may reduce the one-time maximum treatment time to 20 minutes by increasing the efficiency of ultrasonic waves transmitted to the user's body through the ultrasonic pulses of various waveforms described above. This is a significant reduction from 30 minutes, which is the one-time maximum treatment time according to the safety guidelines for low-intensity pulsed ultrasonic therapy devices of the Ministry of Food and Drug Safety and National Institute of Food and Drug Safety Evaluation in Korea, and it is possible to prevent risks such as low-temperature burns of the user and to secure the stability of the internal tissues of the body by reducing the time during which the physical therapy device 100 having the ultrasonic drive device 300 of the present disclosure is in contact with the user's body.

In addition, the ultrasonic drive device 300 of the present disclosure may include a plurality of oscillators 310, and driving of the plurality of oscillators 310 may be controlled by the controller 330. The plurality of oscillators 310 may be disposed in different shapes depending on the body contact area. Consequently, as described above, the ultrasonic waves may be focused onto the treatment area to enhance the therapeutic effect using ultrasonic waves.

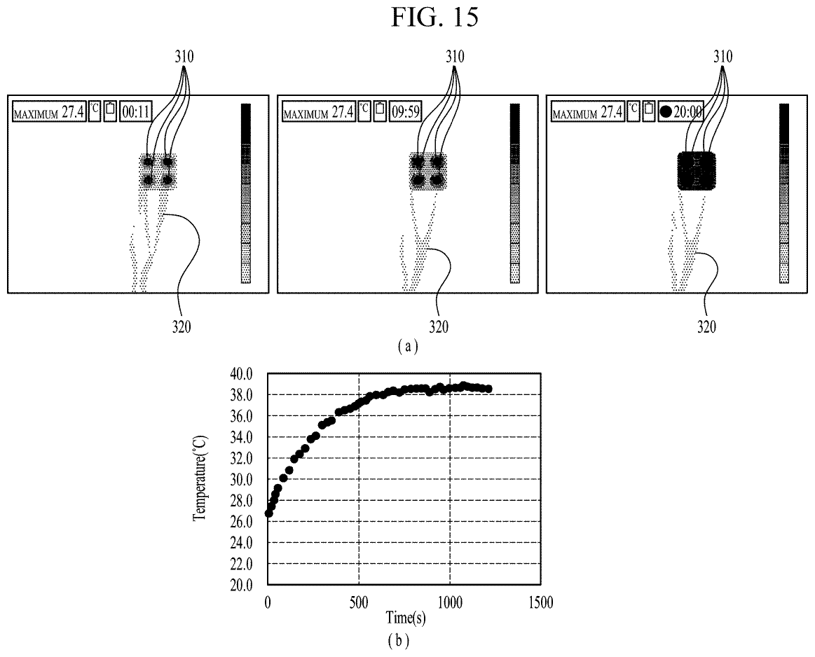

FIG. 15 shows data on a change in the surface temperature of the oscillator 310 over the drive time of the ultrasonic drive device 300 according to the embodiment of the present disclosure.

According to the safety guidelines for low-intensity pulsed ultrasonic therapy devices of the Ministry of Food and Drug Safety and National Institute of Food and Drug Safety Evaluation in Korea, the temperature of all mounting parts that can come into contact with the body must not exceed 43° C. This is to prevent the user's body from being subjected to continuous heat, resulting in low-temperature burns.

The ultrasonic drive device 300 of the present disclosure is set such that the treatment time does not exceed 20 minutes, and when the treatment time elapses, the power of the ultrasonic drive device 300 is automatically turned off by the power supply 331. Therefore, as shown in FIG. 15, the temperature change of the oscillator 310 of the ultrasonic drive device 300 of the present disclosure for 20 minutes was measured by driving the ultrasonic drive device in the state in which the ultrasonic gel is applied to the surface of the oscillator 310.

FIG. 15(a) shows data on the surface temperature of the oscillator 310 at 11 seconds, 599 seconds, and 1,200 seconds acquired using a thermal imaging camera after the ultrasonic drive device 300 is driven, and FIG. 15(b) is a graphical representation thereof. It can be seen that the initial temperature of the surface of the oscillator 310 is 27.4° C., and the temperature of the surface of the oscillator 310 after the ultrasonic drive device 300 is driven for 20 minutes is 39.9° C.

Consequently, it can be seen that the ultrasonic drive device 300 of the present disclosure satisfies the safety guidelines for low-intensity pulsed ultrasonic therapy devices of the Ministry of Food and Drug Safety and National Institute of Food and Drug Safety Evaluation in Korea, since the surface temperature of the oscillator 310 does not exceed 43° C. even after the ultrasonic drive device is driven for 20 minutes.

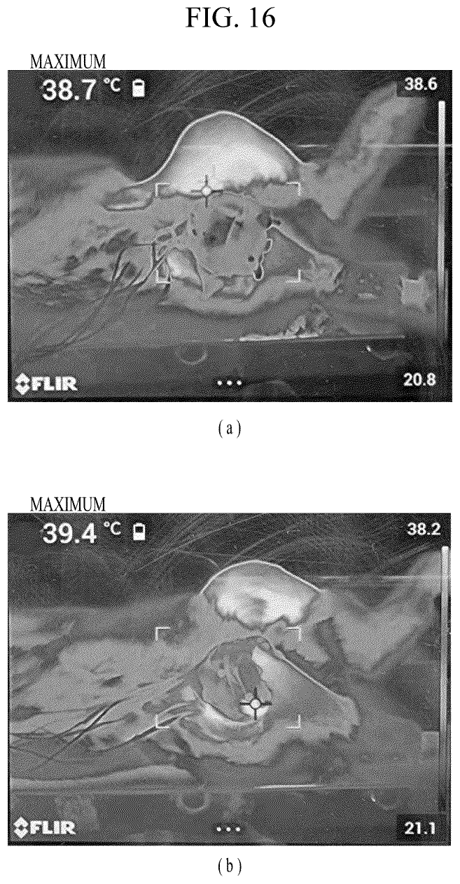

FIG. 16 shows data on a change in the surface temperature over time of one knee of a hind leg of a rabbit to which the physical therapy device 100 having the ultrasonic drive device 300 according to the embodiment of the present disclosure is attached.

According to the safety guidelines for low-intensity pulsed ultrasonic therapy devices of the Ministry of Food and Drug Safety and National Institute of Food and Drug Safety Evaluation in Korea, the initial temperature of the surface of a test target having temperature similar to the human body temperature must be about 33° C., the ambient temperature must be (23±3)° C., and the surface temperature increase must not exceed 10° C.

Therefore, the test target was a rabbit whose body temperature is similar to the human body temperature (about 38° C.), the ambient temperature was about 23° C., and the physical therapy device 100 having the ultrasonic drive device 300 of the present disclosure was driven for 20 minutes in a state of being attached to the right knee of the hind leg of the rabbit. FIG. 16(a) shows data on the surface temperature of the knee of the hind leg of the rabbit acquired using the thermal imaging camera before driving the ultrasonic drive device 300, and FIG. 16(b) shows data acquired using the thermal imaging camera after driving the ultrasonic drive device 300 for 20 minutes. It can be seen that the surface temperature of the right knee of the hind leg of the rabbit increased by 0.7° C. from 38.7° C. to 39.4° C. under the above-described conditions.

Therefore, it can be seen that, even though the ultrasonic drive device of the present disclosure is operated for 20 minutes, the surface temperature rise of the rabbit, the body temperature of which is similar to the human body temperature, does not exceed 10° C., thus satisfying the safety guidelines for low-intensity pulsed ultrasonic therapy devices of the Ministry of Food and Drug Safety and National Institute of Food and Drug Safety Evaluation in Korea.

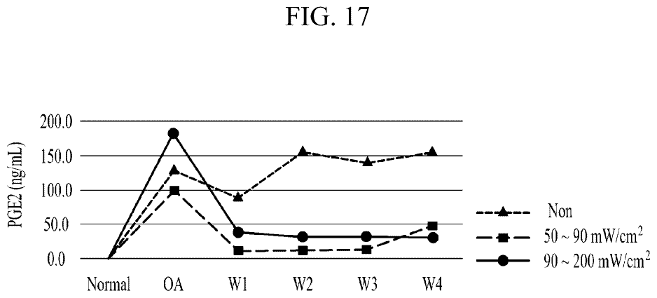

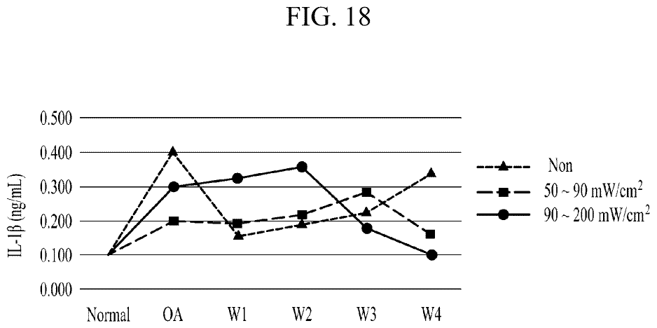

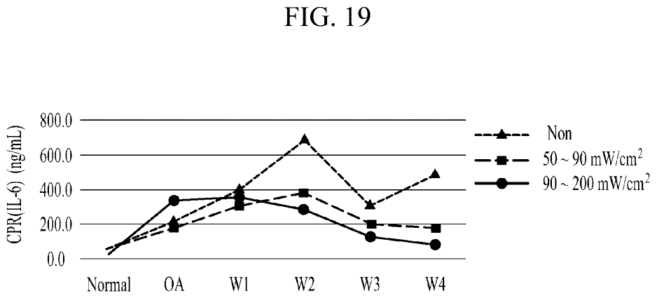

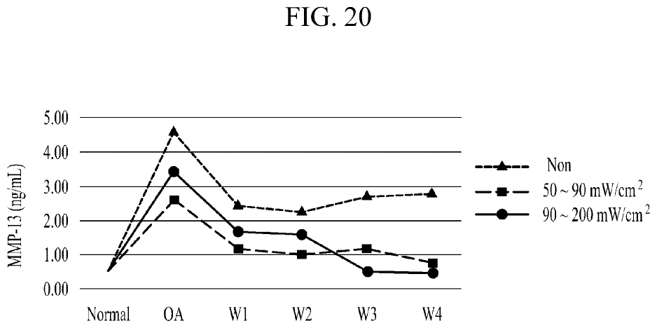

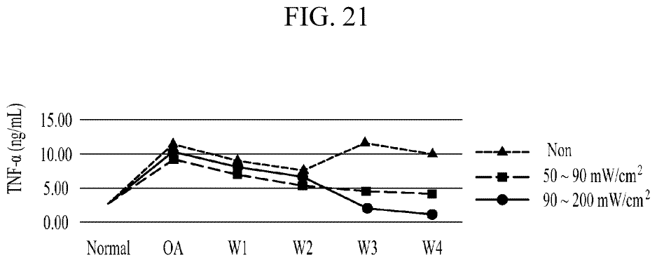

FIGS. 17 to 21 show data illustrating the therapeutic effect of the ultrasonic drive device 300 according to the embodiment of the present disclosure.

In order to investigate the effect of the ultrasonic drive device 300 according to the present disclosure, an animal model of knee osteoarthritis was used to measure changes in the concentration of inflammatory mediators, PGE2, IL-1β, CRP (IL-6), MMP-13, and TNF-α.

Degenerative arthritis, also known as osteoarthritis, is the most common type of arthritis and is characterized by inflammation and pain caused primarily by degenerative damage to the cartilage that protects the joints. Inflammation is an immunological phenomenon in response to external stimuli, and prostaglandin E2 (PGE2), interleukin-1β (IL-1β), IL-6, matrix metalloproteinase-13 (MMP-13), and tumor necrosis-α (TNF-α) produced by inflammatory cells promote the inflammatory response. Here, TNF-α is secreted early in the immune response and promotes early inflammation and progresses to osteoarthritis by promoting proteoglycan degradation and inhibiting resynthesis in the articular cartilage matrix, and IL-1β increases cartilage matrix destruction in osteoarthritis and promotes the production of PGE2, which causes proteoglycan loss and inhibits resynthesis.

Therefore, in order to confirm the therapeutic effect using ultrasonic waves by applying ultrasonic pulses of various waveforms, even though the intensity of ultrasonic waves was reduced to 50 to 200 mW/cm² in the ultrasonic drive device 300 of the present disclosure, the changes in the concentration of inflammatory mediators such as PGE2 described above were measured to determine whether the production of inflammatory mediators was inhibited.

For this experiment, synovial fluid (joint fluid) was collected from the right knee joint of the hind leg of each of three normal rabbits and the concentration of each of the above-mentioned inflammatory mediators was measured. Then, three rabbits underwent surgery to damage the joint by making an incision in the right knee of the hind leg, and the synovial fluid was collected two weeks after surgery to measure the concentration of inflammatory mediators. Then, using a rabbit that did not receive any ultrasonic therapy as a control group, an ultrasonic pulse of 50 to 90 mW/cm² was applied to the knee area where the surgery was performed for 20 minutes once a day for 4 weeks through the ultrasonic drive device 300 of the present disclosure, and synovial fluid was extracted from the right knee joint of the hind leg of a rabbit that received an ultrasonic pulse of 90 to 200 mW/cm² through the ultrasonic drive device 300 of the present disclosure under the conditions described above to measure the concentration of inflammatory mediators.

As a result of the experiments, it was found that, when ultrasonic therapy was performed by applying an ultrasonic pulse having an intensity of 50 to 200 mW/cm² through the ultrasonic drive device 300 of the present disclosure, all of the concentrations of the inflammatory mediators such as PGE2 were reduced compared to the control group. In particular, it was found that the concentrations of the inflammatory mediators were significantly reduced from 2 to 3 weeks after the start of ultrasonic therapy compared to the control group.

It was confirmed from the above results that the ultrasonic drive device 300 of the present disclosure is effective in treating arthritis by suppressing the production of inflammatory mediators, such as PGE2, even if the ultrasonic pulse is applied in the state in which the intensity of ultrasonic waves is reduced to 50 to 200mW/cm².

The above detailed description is to be construed in all aspects as illustrative and not restrictive. The scope of the present disclosure should be determined by reasonable interpretation of the appended claims and all changes coming within the equivalency range of the present disclosure are intended to be embraced in the scope of the present disclosure.

What is claimed is:

1. An ultrasonic drive device comprising:
an oscillator configured to generate ultrasonic waves;
a flexible circuit board electrically connected to the oscillator; and
a controller electrically connected to the flexible circuit board, the controller being configured to supply current to the oscillator and to control driving of the oscillator, wherein
the controller generates an ultrasonic pulse of a first waveform having a first pulse repetition frequency with an intensity of 50 to 200 mW/cm² and varies a duty ratio of the ultrasonic pulse of the first waveform to control the driving of the oscillator.

2. The ultrasonic drive device of claim 1, wherein
the first pulse repetition frequency is 1 to 3 MHz, and
the duty ratio is 20 to 80%.

3. The ultrasonic drive device of claim 2, wherein
the controller controls the driving of the oscillator to generate an ultrasonic pulse of a second waveform having a second pulse repetition frequency of 10 to 100 Hz, and
the ultrasonic pulse of the first waveform is included in the ultrasonic pulse of the second waveform.

4. The ultrasonic drive device of claim 3, wherein
the controller controls the driving of the oscillator to generate an ultrasonic pulse of a third waveform having a third pulse repetition frequency of 0.2 to 1 Hz, and
the ultrasonic pulse of the second waveform is included in the ultrasonic pulse of the third waveform.

5. The ultrasonic drive device of claim 1, wherein
the oscillator is provided in plural so as to be electrically connected to the flexible circuit board, and
the controller controls the driving of the plurality of oscillators.

6. The ultrasonic drive device of claim 5, wherein the plurality of oscillators is disposed in a polygonal shape.

7. The ultrasonic drive device of claim 6, wherein the plurality of oscillators is disposed in a triangular shape on each side of the controller.

8. The ultrasonic drive device of claim 5, wherein the plurality of oscillators is disposed in line in a longitudinal direction of the flexible circuit board while being spaced apart from each other.

9. The ultrasonic drive device of claim 8, wherein the plurality of oscillators is spaced apart from each other by different distances.
